(19) United States
(12) Patent Application Publication
(10) Pub. No.: US 2017/0238879 A1
(43) Pub. Date: Aug. 24, 2017

(54) Method of Analyzing the Brain Activity of a Subject

(71) Applicant: Assistance Publique - Hopitaux de Paris, Paris (FR)

(72) Inventor: Denis Ducreux, Le Chesnay (FR)

(73) Assignee: Assistance Publique - Hopitaux de Paris, Paris (FR)

(21) Appl. No.: 15/518,844

(22) PCT Filed: Oct. 13, 2015
(86) PCT No.: PCT/EP2015/073685

§ 371 Date: Apr. 13, 2017

(30) Foreign Application Priority Data
Oct. 14, 2014 (FR) 1459862

(51) Int. Cl. A61B 5/00; A61B 5/16; A61B 5/0476; A61B 5/04; A61B 5/055; A61B 6/03

(52) U.S. Cl. CPC A61B 5/7264; A61B 5/055; A61B 6/037; A61B 5/0476; A61B 5/04012; A61B 5/4076; A61B 5/164

(57) ABSTRACT

The invention concerns a method of analysing the brain activity of a patient performing a given task or in response to an external stimulus, by comparison of standardized data with data in a database, by means of fuzzy logic algorithms.

The invention relates to the analysis of the scope of brain activity of subjects, and to the implementation of methods and procedures that make it possible to determine the activity thereof in response to stimuli or during the performance of specific tasks.

The analysis of brain operation is one of the great issues of the 21^{st} century. By understanding how the “healthy” brain works, it is possible to envisage developing new therapies making it possible to improve the functional capacities of patients exhibiting neurological deficiencies or psychiatric disorders and/or to determine the effectiveness of treatments for these patients. Moreover, such techniques could make it possible to detect a brain activity in patients incapable of interacting with the environment (such as patients in a coma) and predict any sequelae or a capacity to change.

Thus, over 30 years the neurosciences have made major advances in the analysis of cognitive processes, on the one hand through the knowledge acquired in neuro-anatomy, but also thanks to the advances made in neuro-computing (neural networks, artificial intelligence, etc.) and in neuro-imaging (particularly the advances in functional Magnetic Resonance Imaging).

Nevertheless, even though it is now possible to study the differences in functional brain areas when tasks are performed, for example, between healthy subjects and patients affected with neurological or psychiatric pathologies, it is difficult to be able to accurately categorize the neural networks activated during different tasks given how variable these networks can be from one subject to another.

The studies currently performed are validated to the subject group scale, and do not take account of the individual differences which do however characterize the uniqueness of the individual, nor do they take account of the temporal variability of the activated brain areas.

Seghier et al (Neuroimage, 2007, 36, 3, pp. 594-605) disclose a new method for analyzing the brain activity of a subject during the performance of a task or in response to a stimulus comprising the analysis of grey matter data obtained by functional MRI.

US 2004/092809 describes a computer-assisted method for diagnosing a condition of a subject in which this condition is associated with an activation in one or more regions of interest, the method comprising:
having the subject perform a task or have a perception, capable of selectively activating one or more regions of interest associated with the condition;
measuring the activity of the region or regions of interest only when the task is performed or when the subject has the perception;
diagnosing the condition associated with one or more regions of interest on the basis of the activity in response to the behavior or the perception;
executing an intervention (applying a pharmacological agent or performing a therapeutic method);
repeating this process one or more times, including repetition of the behavior, measurement of the activity and diagnosis at a subsequent moment;
observing the changes between the measurements, associated with the intervention.

It is therefore necessary to benefit from a method which can provide a good analysis of the brain activity of a subject in response to a stimulus, or during the performance of a given task, and which takes account of the variability which exists between the different people.

The applicant proposes using the properties of fuzzy logic algebra to analyze such brain activities.

Unlike Boolean algebra, fuzzy logic makes it possible to measure similarities between a state and reference states. In conventional Boolean algebra, such a comparison makes it possible to obtain only one or the other of the values of the {true, false} pair. In fuzzy logic, there are degrees in the satisfaction of a condition, which are represented by percentage of similarities.

Fuzzy logic is thus used in many fields such as automation (ABS brakes, process control), robotics (shape recognition), road traffic management (red lights), air traffic control (air traffic management), the environment (meteorology, climatology, seismology, lifecycle analysis), medicine (diagnostic assistance), insurance (selection and prevention of risks).

Fuzzy logic makes it possible to compare complex elements to reference elements, and to determine percentage similarities between the input element and the elements of the reference base, and to draw conclusions on the nature of the input element.

For example, if the input element exhibits
80% similarity with the reference element X1 corresponding to the state E1,
75% similarity with the reference element X1 corresponding to the state E1 and
50% similarity with the reference element X3 corresponding to the state E2,

it will then be possible to conclude therefrom that the input element corresponds very probably to a representation of the state E1. Percentages can thus be computed to determine the probability for the input element of effectively representing the state E1 (these percentages depend in particular on the percentage similarity with each of the reference elements representative of the state E1).

As seen above, there are a large number of fuzzy logic algorithms that make it possible to compare complex input data with reference data, and to compute similarities with reference events. In particular, such artificial intelligence algorithms based on fuzzy logic are used to detect plagiarisms, notably in universities.

New algorithms can also be easily developed to address particular issues, the rules underpinning fuzzy logic having been formalized by Lotfi Zadeh as early as 1965.

The applicant therefore proposes to use these fuzzy logic algorithms in the analysis of the brain activity of a subject, measured during the performance of a task or in response to a stimulus. In a particular embodiment, said stimulus is an absence of stimulus (resting state).

The principle of the method that is the subject of the present application is to:
normalize the data acquired during the performance of the task or in response to the stimulus
compare these normalized data with data present in a database, by using the principle of fuzzy logic (determination of similarities between input data and data in the base)
based on the similarities, automatically determine the individual variations, and identify, in the subject, the discrepancy or the concordance between the brain activity for a task performed by the subject and the brain activity as measured in other subjects and represented by the data present in the base.

Thus, the invention relates to a method for analyzing the brain activity of a subject during the performance of a task or in response to a stimulus comprising the steps of
a. normalization of data (d1) collected during the establishment of said task or application of said stimulus in order to obtain normalized data (d2)
b. comparison of said normalized data (d2) with data (d3) present in a normalized database

said data (d3) of said database each being specific to a task of a given stimulus,

said comparison being performed by a fuzzy logic algorithm, said method making it possible to determine a degree of similarity of said normalized data (d2) with data present in the normalized database, said method making it possible to determine the brain activity of said subject during the performance of said task or in response to said stimulus.

The technical effect obtained by the method thus described is the capacity to be able to analyze complex data and to be able to draw a conclusion on the similarity of these data with reference data. This conclusion is drawn from the percentage similarity between the data (d2) and the data present in the base and representative of a task or a given stimulus, percentage computed by the fuzzy logic algorithm implemented in the method. According to the similarity with the data representative of a given task or stimulus, it will be possible to conclude on the “normality” of the activity of the patient in the performance of this task or the response to this stimulus, or on differences, which therefore possibly reflect a psychiatric or neurological deficiency. If the task or the initial stimulus is not known, the percentage similarity will be able to make it possible to characterize this task.

The determination of the brain activity should be understood to be the determination of the areas activated in the brain during the performance of the task or the application of the stimulus, but may also incorporate the temporal variation of activity of the areas of the brain during the performance of the task or the application of the stimulus. It is known that the activated areas which can be determined, during the performance of the task or the application of the stimulus, are grey matter areas.

The database contains a certain number of data. Each datum (d3) present in the database is specific to a task or to a given stimulus. However, the database may contain several data (d3) for a task or a given stimulus. This is even preferable, because that will make it possible to improve the accuracy of the analysis of the input data and the conclusion which will be able to be drawn.

The method can be used in many fields of application: Comparative analysis of the state of wakefulness at rest among healthy subjects and among patients who cannot communicate with the environment (such as patients in a coma)

Said brain activity reflects the cerebral response of said subject to one or more external stimuli. It notably concerns performing a comparative analysis of the state of wakefulness at rest among healthy subjects and among patients in a coma, by subjecting said subjects to varied stimuli (listening to music, sensation of touch on various parts of the body, stimulation by speech, etc.).

The reference data of the database are those obtained on healthy subjects, for the same stimuli.

According to the response of the patient to the stimuli and the comparison with the data obtained for the healthy patients, it may be possible to determine the capacity of the patient to respond to certain stimuli, which may make it possible to characterize the depth of the coma (deep, semi, moderate), or could serve to establish a recovery prognostic index.

Thus, in this case, if a brain activity of said subject is observed that is similar to the brain activities observed for healthy subjects, this will be more of a favorable marker of change or will make it possible to determine a certain state of consciousness in the subject being studied.

Comparative Analysis of Psychiatric Problems

In this mode of application, said brain activity will reflect the response given by said subject to specific tasks linked to psychiatric problems (such as depression, schizophrenia, autism, etc.).

The specific tasks may be tasks linked to olfaction and/or memory for depression, tasks linked to mental calculation for autism, or requests for mental imaging representation for schizophrenia.

The comparison is performed with data obtained for healthy subjects performing the same tasks.

The percentage similarity observed for a given task could make it possible to classify the depth of the problems, and/or give a prognosis on therapeutic effectiveness during treatment with medicines (return to “normal” data).

It should be noted that, in this case, it is to be expected that the brain activity of said subject is to exhibit significant variations with the brain activities observed for healthy subjects, and that the method therefore makes it possible to identify variations characteristic of the psychiatric problem considered. This then makes it possible to make diagnoses or to check the effectiveness of treatments.

Comparative Analysis of Neurological Problems

In this embodiment, said brain activity reflects the response given by the patient during a clinical and functional evaluation of a deficiency or of a neurological problem (such as a cerebral vascular accident, a tumor, multiple sclerosis or any degenerative pathology, etc.).

The tasks performed are notably tasks related to motricity, language, or memory.

The percentage similarity is computed on the basis of the comparison with data from healthy patients, having performed the same tasks. It should make it possible to give a functional recovery prognostic index in the short and medium term among these patients, and a prognostic index of therapeutic effectiveness.

It should be noted that, in this case, it is to be expected that the brain activity of said subject exhibits significant variations with the brain activities observed for healthy subjects, and that the method therefore makes it possible to identify variations characteristic of the psychiatric problem considered. This then makes it possible to make diagnoses or check the effectiveness of treatments.

Lie Detection

In this embodiment, the objective is to detect whether the subject tells the truth or lies in responding to different questions.

In this case, the data are compared to the data generated by reference people, of whom it is known if they have told the truth or lied in responding to questions.

The brain activity is, indeed, different when a subject tells the truth or lies. The data studied will therefore be different depending on the veracity of the responses of the subject.

The percentage similarity with the data observed for the people speaking the truth or the people lying will make it possible to define a probability of lies or truth for the subject studied, according to the questions posed.

Neuromarketing

In this embodiment, the brain activity measured reflects a qualitative reaction of subjects after application of stimuli. This qualitative reaction can notably be understood as an I like/I do not like reaction.

The data of the base, with which the input data are compared, are data obtained among subjects exhibiting positive or negative reactions upon exposure to agreeable or disagreeable stimuli. Indeed, the brain activity is different in cases of positive or negative sensation.

The implementation of this embodiment offers applications in the field of neuromarketing, for better understanding the reactions of subjects upon the presentation of new products, or the evocation of product development.

Resting State

In this embodiment, the brain activity measured is that of the subject in the absence of any explicit task requested of the patient, or of application of external stimulus.

There are therefore many applications of the method described above.

The data (d1) collected during the performance of the task or the application of the stimulus are notably MRI, PET scanner, echography or electroencephalography (EEG), optical imaging data.

The data (d1) therefore correspond to the signals collected during the performance of the task or the application of the stimulus. However, it is not a priori possible to use these raw data, which are very dependent on the subject studied. It is therefore necessary to normalize these data. This normalization is performed by methods known in the art in order to eliminate the inter-subject variability (in particular the size of the brain). The normalized data can then be easily compared to the reference data, also having undergone the same normalization, by the fuzzy logic algorithms, to obtain similarities between the “test” normalized data and the reference data.

It should be clearly understood that the data (d1) are signals which represent levels of activation of different areas of the brain during the acquisition time of these signals (in particular the time of performance of the task or of application of the stimulus).

Thus, electroencephalography (EEG) measures the electrical activity of the brain by electrodes placed on the scalp.

However, it is preferred when the data (d1) acquisition method is MRI.

In particular, the data (d1) collected during the performance of the task or the application of the stimulus are data obtained by functional MRI and therefore representing the activity of the grey matter during the performance of the task or the application of the stimulus.

Activation functional MRI (fMRI) is a routine technique for exploring brain functions. The principle relies on the computation, in real time, of the oxygen expenditure linked to the activity of the cerebral cortex, in response to the performance of a cognitive task (language, motricity, tactile or visual stimulation, memory, etc.) or of a stimulus. It consists in recording minimal local cerebral hemodynamic variations (variation of the properties of the blood flow), when these areas are stimulated.

In this embodiment, it is also preferable for said data (d1) to further comprise MRI data making it possible to representing the white matter fibers of the brain of said subject.

These data are obtained by diffusion MRI, which makes it possible to compute, at each point of the image, the distribution of the directions of diffusion of the water molecules. Since this diffusion is constrained by the surrounding tissues, this imaging modality makes it possible to indirectly obtain the position, the orientation and the anisotropy of the fibrous structures, in particular the bundles of white matter of the brain. That makes it possible to see the water which flows along the fibers. Even though diffusion MRI is currently the only technique which makes it possible to observe the in vivo brain connectivity non-invasively, the use of other techniques making it possible to obtain the same result, if developed in the future, would also be perfectly appropriate.

In this embodiment preferably, morphological data are also acquired by MRI.

Morphological MRI, reference examination in neuroradiology, allows for a precise anatomical analysis in three dimensions, and in particular makes it possible to place functional images.

However, any other morphological imaging method (in particular radiography, scanner, echography) can also be used in the context of the data (d1) acquisition method. Those are imagings which take a photo of the organism but without studying their operation as in functional imaging.

It is recalled that the signal in MRI is weak and must be accumulated by repeated stimulations. This is done during sequences defined by certain parameters according to the selected disturbance. The duration of a sequence is variable and currently lasts between 0.5 and 15 minutes.

The stimulations are generally repeated with a period of 1.5 s, that is to say that data are recovered every 1.5 s.

A method is described below that makes it possible to normalize MRI data obtained for the implementation of the method for analyzing the brain activity described above.

However, it is important to recall certain rules and definitions which will make it possible to better understand the method described below.

During the performance of a given task, the brain will exhibit an activity, which is materialized by the sequential and/or concomitant activation of various grey matter areas.

However, it is possible for some other areas not involved in the performance of the task to also be identified (by functional MRI) as being activated in the performance of this task.

The Applicant therefore proposes also assessing the bundles of white matter to determine whether the areas detected as activated are linked to one another. In effect, one can consider that an area activated independently (not linked to other areas activated during the performance of the task) is not in fact linked to said task.

There are already reference brain atlases, available in particular at the Montreal Neurological Institute (MNI) which make it possible, by co-registration, to normalize anatomical MRI and functional MRI data, and to superimpose the two normalized images thus obtained. It is thus possible to use software available in the art, like the FLIRT software (FMRIBs linear image registration tool—linear inter- and intra-modal registration) developed by the members of the FMRIB (Functional Magnetic Resonance Imaging of the Brain) analysis group, Oxford University, Great Britain.

Moreover, it is also possible to construct an atlas of the networks of white matter fibers, that can be normalized also on the same basis as the MNI atlas. Such a normalized atlas on the database present at the

MNI or on the Talairach atlas is described by the “Laboratory of brain anatomical MRI” of the Johns Hopkins Medical Institute.

http://cmrm.med.jhmi.edu/cmrm/atlas/human₁₃ data/file/Atl asExplanation2.htm.

Thus, the use of known techniques will make it possible to superimpose, in a normalized manner, the anatomical, functional (grey matter) and structural (white matter) data.

There are two ways of conducting a data acquisition by MRI (or any other method) from a subject performing a task or subjected to a stimulus.

As seen above, the functional data (activation of the areas of grey matter) are generated by functional MRI. It is recalled that functional MRI consists in recording minimal local cerebral hemodynamic variations (variation of the properties of the blood flow), when these areas are stimulated. The location of the activated brain areas is based on the BOLD (Blood

Oxygen Level Dependent) effect, linked to the magnetization of the hemoglobin contained in red blood cells.

In order to meet the constraints of temporal resolution and of T2* sensitivity (relaxation time T2* of the hydrogen nuclei of the water), the functional MRI sequences are generally of ultra-fast echo planar (EG-EPI) type, with matrices of small size (and therefore a low spatial resolution). The BOLD contrast obtained is very low (variation of the signal by a few percent only), so it is necessary to repeat the acquisitions in time, during different activation tasks, to produce a statistical comparative study of correlation between variations of the signal measured in each voxel and variations of the tasks. The activation differences will thus be related to the difference between the two tasks.

The sequence of the tasks and their mode of repetition constitute the activation paradigm. It comprises at least one reference task, and another task in which the sole difference corresponds to the activity that is to be studied.

The term paradigm is employed to express the way in which the trial protocol was designed and conceived in its broad outlines.

Two types of paradigms are thus defined, which will be chosen, according to the stimulus or the task.

Block paradigm: the activities are organized in blocks of a few tens of seconds which alternate at regular intervals. Within the same block, the hemodynamic responses will overlap and accumulate before forming a plateau.

Event-driven paradigm: the activities or stimuli are unique or presented in short repetitions, with a sequencing which can be pseudo-random (which avoids the anticipation phenomenon), and with possible measurement of the performance of the response (delay and precision of the response, etc.). The local hemodynamic response is thus evaluated during the different activities. The time response to each stimulus is recorded and averaged over several events.

It is also possible to work (implement the methods described in the application) by using data acquired when the subject is “at rest” (resting state), that is to say in the absence of stimulus or in the absence of any task assigned to the subject. In this embodiment, the regional interactions which are produced when the subject does not perform any explicit task are evaluated. In this embodiment, the comparison of the data with the data contained in a database (see later) makes it possible to determine a posteriori the brain functions performed or implemented by the subject during the acquisition of the data. Such an approach has notably been described by Shimony et al (Acad Radiol. 2009 May; 16(5):578-83).

For the motor activities it is possible to take the resting state as reference activity, and a repeated movement of the fingers as activity. For the cognitive activities (language, interpretation, memory, etc.), the protocols are more complex and designing relevant tasks may prove more difficult, although these protocols are now used routinely in the art.

It is also possible to simultaneously record, during the examination, information on the responses of the patient (frequency of movements, delay to respond to a stimulus, correct or incorrect response, etc.) which are incorporated in the statistical analysis model.

The functional MRI data analysis technique is known. It is notably described by Hoa (http://www.imaios.com/fr/e-Cours/e-MRI/irm-fonctionnelle-cerebrale, reproducing Irm Pas a Pas (Edition Noir & Blanc)).

Preprocessing: the images are smoothed to reduce the noise and the artefacts (movements, orientation and spatial distortion) are corrected.

Normalization: it is necessary to compare examinations of different patients or performed at different moments. The images are realigned either between two examinations, or relative to a reference atlas (NIM, Talairach), to make them superimposable, in the same spatial coordinate.

Statistical analysis: it is based on a mathematical modeling of the expected hemodynamic response, which depends on the paradigm employed. The type of model most commonly used is the generalized linear model (GLM). This model will serve to detect, voxel by voxel, those for which the signal variation in time is linked to the sequence of the different activation tasks. The pixels considered statistically significant can then be represented by superimposition on a high-resolution morphological imaging in order to be better located.

Finally it should be recalled that the MRI data acquisition period (interval between two acquisition moments) is generally of the order of 1.5 s. However, the brain responses are more of the order of a hundred or so milliseconds.

The aim of the MRI data processing method described hereinbelow is the generation of normalized data (d2) which can be used in a method for analyzing brain activity described hereinabove.

This method also makes it possible to generate genuine maps of brain activity during the performance of the task or the application of the stimulus, identifying not only the activated brain areas, but making it possible to determine the relationships between them by displaying white matter fibers linking the different brain areas. By extension, these maps will be qualified as “GPS” maps, because they provide both the areas of functional activation of the brain, but also the “routes” (bundles of white matter) linking these areas.

The method is based on the following sequences:
normalization of the MRI data (anatomical, functional and structural) for these data to be all in the same normalized coordinate
processing of the normalized functional MRI data.

Interpolation of the Data to Improve the Temporal Resolution

Optionally, but preferably, interpolation of the data between two acquisition times (for example, change from an interval of 1.5 s to 10 intervals of 150 ms to better reflect the physiological brain response times): the data are interpolated by applying the general linear model (or generalized linear model).

Identification of the activated grey matter brain areas and construction of a mapping using the connectivity of the bundles of white matter

For each temporal block (each temporal acquisition block, or each temporal block generated upon the amplification of the optional step specified above if implemented), the analysis is conducted voxel by voxel, to identify the activated functional brain areas, then one looks for the existence of any bundles of white matter fibers linking these activated brain areas. The map of the bundles of white matter fibers has been produced before or after the performance of said cognitive task or the application of the stimulus, since the analysis during the application of the stimulus generally makes it possible to detect only the activated areas of grey matter.

The brain activity linked to a task is in fact represented by a sequence of activations of interconnected brain areas. The superimposing of the functional atlases and the structural atlases and the looking to see if there are fibers linking the activated areas make it possible to define whether these areas are linked and deduce therefrom this sequence linked to the task or to the stimulus. Moreover, if no bundle of white matter linking an activated area and the other areas is identified, it can be assumed that this “orphan” area is not involved in the performance of the task or the response to the stimulus. This therefore makes it possible to reduce the false positives.

Computation of a global value of correlation with the paradigm for each of the activated areas

The following step consists in assigning, for each area of grey matter, a global value of correlation with the paradigm. For this, a geometrical average of the correlation coefficients of each of the voxels of the area considered can be calculated to obtain a global value for the area considered. The computation is done for each temporal block.

Thus, as indicated by Rohmer et al (Detection of Brain Activity by fMRI (EPI) using a Region Growth Algorithm; seventeenth GRETSI symposium, Vannes, 13-17 September 1999), “since the MRI scanners make it possible to rapidly acquire a set of images (a brain volume in under 10 seconds), the solution to this weak signal variation consists in increasing the temporal resolution of the sequence. Then, not only are two values characterizing the resting state and the activation state associated with each voxel, but also a temporal signal. The aim is therefore to be able to characterize the temporal trend of each voxel while the subject performs a task according to a very specific paradigm. Most of the work linked to fMRI then consists in analyzing the temporal sequences associated with the voxels to determine the state of activity of a region of the brain.

[Bandettini et al (Processing Strategies for Time-Course Data Sets in Functional MRI of the Human Brain, Magn. Reson. Med., 1993, vol. 30, p. 161-173) presented] a method which considers that the temporal signal associated with a voxel has to be strongly correlated with the signal of the paradigm to represent an area of activity. To obtain a mapping of brain activation, the coefficient of linear correlation between paradigm and temporal signal is therefore computed for each voxel. The higher the correlation coefficient, the more active the area”.

It should be noted that this step of computation of the average activity of an area considered can be performed over the whole set of the areas of grey matter mapped in the brain, but it is favorable (in particular for issues of optimization of the computation means) to perform this work only on the areas identified as having voxels activated in the preceding step. The reduction of the number of areas to be analyzed makes it possible to reduce the RAM memory requirement.

At the end of this step, there is obtained, for each temporal block, a unique coefficient of correlation of said area with the paradigm, the value of which represents in particular the intensity of activation of the area.

This step of averaging by area of activation thus makes it possible to greatly reduce the size needed to store the data. In fact, the initial data comprised the set of the factors of correlation of each voxel with the paradigm (i.e. of the order of 10⁷ voxels after acquisition and normalization on the MNI atlas), whereas the data obtained after averaging represent the factors of correlation of each area of activation pre-mapped in the atlas with the paradigm (for example 116 areas only, described in FIG. 1).

Reduction of the Complexity by Grouping Together the Data Corresponding to the Same Actions of the Paradigm

It is then possible to further reduce the size of the data by performing a reduction of temporal dimension, which corresponds to an averaging of the data thus computed and which corresponds to the same stimulation, or to the same state of performance of the task within the paradigm.

By way of illustration, in the case of a block paradigm, the sequence Resting (30 seconds)/Stimulus (30 seconds) is repeated three times.

The data processing operations are performed as described above.

There are then obtained three sets of data representing the brain activity of each brain area (a map for each of the three blocks of the paradigm).

It is then possible to average the three sets of data into just one which represents the “stimulus” activity sequence of the paradigm.

Such a reduction of the complexity on the event-driven paradigms can also be performed by clearly identifying the events.

It should be noted that the data obtained make it possible to build maps by using market-standard graphic representation software. These maps thus obtained are maps in four dimensions (spatial and temporal) covering the blocks of activity of the paradigm and making it possible to see the activated brain circuits and the variations and sequences of activation during each activity block of the paradigm. The anatomical data make it possible to see the shape of the brain and it is possible to represent the various areas which are activated over time during the performance of the task (with color codes making it possible to reflect the level of activity), as well as the paths (white matter fibers) linking these areas.

They are thus space-time maps, of the brain activity of a subject, showing the activated areas, as well as the functional neural networks, during the performance of a given task or in response to a stimulus. As indicated above, these maps can be qualified as “GPS” maps.

It should also be noted that, these maps having been produced on normalized data can therefore easily be compared to one another of the artificial intelligence software systems based on fuzzy logic, as described above.

It is clear that the above methods are preferentially implemented by computer.

The invention thus relates to a method for generating normalized data that can be used for the implementation of a method for analyzing brain activity, as described above, in which said data (d1) to be normalized comprise, for each temporal acquisition block (t1) of said data (d1) during the performance of the task or the application of the stimulus, the protocol for performing said task or for applying said stimulus representing a paradigm:
a. functional brain data of the grey matter obtained during the performance of said cognitive task or after application of the stimulus
b. structural data of the white matter fibers linking the grey matter areas obtained during the performance of said cognitive task or after application of the stimulus
c. anatomical data of the brain of said subject, said method comprising the steps of
i. normalization of said anatomical data, in order to represent the brain of the subject in a normalized coordinate
ii. normalization of said functional data on the basis of a normalized functional atlas representing the cortical areas and the central grey nuclei of the brain, said functional atlas being in the same normalized coordinate as the atlas of (i)
iii. normalization of said structural data of the white matter fibers on the basis of said anatomical atlas of (ii)
iv. optionally, increasing the temporal resolution between each temporal acquisition block (t1), by dividing into equal parts (interpolated temporal blocks t2) the time between two temporal acquisition blocks (t1) and interpolating the statistically significant signal variations at the level of each voxel acquired for the functional data by using the generalized linear model
v. for each temporal block t1 or, in the case of implementation of the step iv, for each temporal block t2, searching, voxel by voxel, for the activated functional brain areas, and for the bundles of white matter fibers uniting each of these activated brain areas
vi. for each of the functional brain areas studied, averaging the correlation coefficients of each of the voxels of said area with the paradigm, in order to obtain a unique correlation coefficient of said area with the paradigm
said normalized data (d2) obtained on completion of the step vi this consisting of:
for each temporal block t1 (or for each temporal block t2 in case of implementation of the step iv)
   normalized data representing the white matter fibers of the brain of the subject
   normalized data representing the correlation coefficient of each functional area (grey matter) with the paradigm (performance of the task or application of the stimulus)
   definition of the task performed or of the stimulus applied.

The normalization of the anatomical data can notably be performed by co-registration on the atlas T1 of the MNI, by using the FLIRT software described above.

The normalization of the functional data can be performed on the basis of a normalized functional atlas representing the cortical areas and the central grey nuclei of the brain, in particular the 116 brain areas described in FIG. 1. This functional atlas can be written in the same normalized coordinate as the atlas T1 of the MNI. There is, at the MNI, a functional atlas which is in the same coordinate as the atlas T1.

The normalization of the structural data of the white matter fibers can be performed on the basis of any existing atlas, in particular the atlas of the John

Hopkins Medical Institute described above. Alternatively, these structural data can be normalized on the basis of a normalized atlas in the same coordinate as the atlas T1 of the MNI, and showing the 58 white matter fibers described in FIG. 2.

In a particular embodiment, said functional brain areas areas studied in the step vi (for which an average of the correlation coefficients of each voxel present in each zone is calculated) are only the areas that have been previously selected after a voxel-by-voxel search has identified that they are activated.

This step vi is performed by geometrical averaging of the values of the correlation coefficients of each of the voxels of the brain area for each temporal block t1 or t2.

It is however possible to weight, for each voxel, the correlation coefficient value used in this geometrical average, notably by watching, for the brain area considered, the maximum, minimum, average value and standard deviation of the correlation coefficients and not taking account of the voxels having a value below a threshold determined from this information (it is possible, by way of illustration, to not take account of the correlation coefficient voxel values below the average—two times the standard deviation).

It is possible
to compute these maximum, minimum, average value and standard deviation of the correlation coefficients for each temporal block in each paradigm block, and perform this weighting on these values within each paradigm block, or
“to align” the different blocks of the paradigm (there is a repetition of the tasks or the stimuli, therefore these blocks can be aligned) and perform the weighting described above from the maximum, minimum, average value and standard deviation of the correlation coefficients for the set of the temporal blocks t1 or t2 placed at the same point in each of the blocks of the paradigm.

This step of averaging by area of activation thus has the technical effect of greatly reducing the size needed to store the data. In fact, the initial data comprised the set of the factors of correlation of each voxel with the paradigm, whereas the data obtained after averaging represent the factors of correlation of each area of activation pre-mapped in the atlas with the paradigm (for example 116 areas only, described in FIG. 1.

In a particular embodiment, a step of reduction of the temporal dimension of said map is additionally performed by averaging the results for each of the blocks of the paradigm (values of activation of each of the activated areas) corresponding to the steps of action or of stimulus within the paradigm.

This step of temporal reduction thus uses the fact that, in a block paradigm, the same task is repeated several times or the same stimulus is applied several times. The performance of this step therefore makes it possible to obtain a single map of the brain activity of the patient considered during the performance of the task or in response to the stimulus. By not implementing this step, it is possible to retain a number of maps equal to the number of blocks of the paradigm.

The technical effect of this step is to reduce the memory needed to store and analyze the data.

It is also possible to perform another step of temporal reduction by reducing the temporal resolution within the map. It is recalled that, generally, the temporal resolution has been increased because of the low temporal resolution during the acquisition of the primary brain data (change from a resolution of 1.5 s (i.e. a gap of 1.5 seconds between each data acquisition) to a resolution of 150 ms) by an interpolation.

This other temporal reduction step corresponds in fact to the reverse operation, that is to say to calculating an average of a predetermined number of temporal blocks t1 or t2, in order to reduce the number thereof.

The aim is always to reduce the computation power and memory needed for the storage and the manipulation and analysis of the data.

In conclusion, the implementation of the method overall makes it possible to obtain normalized data consisting of
normalized data representing the white matter fibers of the brain of the subject (for example the 58 bundles of white matter if using the data of FIG. 2).
Normalized data representing the brain activity (correlation coefficient) of each functional area (grey matter) during the performance of the task or application of the stimulus (for example over the 116 areas of grey matter of FIG. 1).
Definition of the task performed or of the stimulus applied.

In the case of the application in the absence of any stimulus or task assigned, this last normalized data mentions it, that is to say specifies that the patient is in the resting state. Thus, this resting state is considered in the same way as when a stimulus is applied or a task is assigned.

These normalized data can be stored in a database. This database will preferentially be constructed in such a way that it will have three inputs:
an input corresponding to the task or to the stimulus applied
an input corresponding to the map (anatomical, functional, structural and temporal data)
an input corresponding to a weighting coefficient.

The principle of the weighting coefficient is as follows: the “GPS” maps generated above are representations of the brain activity of a subject during the performance of a task or upon reception of a stimulus.

However, and as seen above, given the complexity and the variability between two subjects of the brain connections, the “GPS” maps of two different subjects will show differences.

However, the greater the number of maps present in the base for a task or a given stimulus, the more it is possible to determine the activation sequence that is most likely or representative of said task or stimulus.

Consequently, each of the maps present in the base will be weighted by a weighting coefficient, computed by comparing the set of the data of each map with one another, and by determining their similarity threshold, notably by an algorithm based on fuzzy logic.

Thus, the database is an interactive base, which evolves each time a new map is stored therein. The more different inputs the database has for a same task, the more accurate the weighting coefficient of the new input will be. The database therefore becomes increasingly relevant as it is enriched with new inputs.

When the task or the stimulus associated with this new map is known, said new map is compared to the set of the maps which are already present in the database by the method described above. It is then possible to compute a weighting coefficient for this new map, and recompute the weighting coefficients of the other maps.

When a map is introduced without knowing the task or the associated stimulus (particularly in the cases of detection of response truth or neuromarketing), said new map is compared to the set of the maps present in the base, or to one or more subsets of said base (in particular, when searching to see if a person is telling the truth or lying, the comparison is made to the maps associated with the “truth”/“lie” tasks).

The percentage of similarity obtained after these comparisons makes it possible to conclude as to the probability that the person has or has not performed a given task.

For each given task or stimulus, it is also possible to create a “reference” map, from the set of the maps of the database relating to this task or this stimulus.

This reference map is notably created by calculating an average of the maps different maps, weighted by the weighting coefficients.

By way of illustration, a first map is input for a given task with a weighting coefficient, which is 1.

After analysis of the activity of another subject having performed the same task, the new map is input into the base.

The two inputs are compared (grey and white matter) by fuzzy logic software. A global percentage similarity can be deduced.

1/1=100%

2/1=80%

Each input 1 and 2 will have the same weighting coefficient (it is not possible to define which is the more “fair”) in the single input (reference map).

Then, data generated on a third subject are input, and the percentage similarity is computed by fuzzy logic software.

3/1:70%

3/2=90%

The reference map, in which the weights of the maps 2 and 3 will be greater than the weight of the map 1, is then recomputed.

When this reference map is created for each of the tasks or stimulus, it is then of certain use when in possession of a map without knowing the task or the associated stimulus. It is then possible to compare the new map with the set of the reference maps to identify the reference map or maps most similar to this new map and make a second comparison with the maps corresponding to this or these reference maps. Such is in particular the case when evaluating the maps obtained for patients in resting state.

In the embodiment described above, the database is considered to contain the “GPS” maps obtained from functional brain data of the grey matter, obtained by activation functional MRI, structural data of the white matter fibers, obtained by diffusion tensor MRI, normalized on the basis of anatomical data obtained in T1-weighted volume morphological MRI.

However, the principle of construction of the database, in which weighting coefficients are given to the various data incorporated in the base, computed by comparing the set of the data with one another (for the same task), and by determining their similarity threshold, notably by an algorithm based on fuzzy logic, is applicable for any type of data as generated by any other type of measurement (in particular PET scanner, echography, electroencephalography (EEG), or optical imaging), after normalization.

The database will preferentially be constructed such that it will have three inputs:
an input corresponding to the task or to the stimulus applied
an input corresponding to the normalized data
an input corresponding to a weighting coefficient.

This database is used in the comparison on the basis of the fuzzy logic algorithm, as described above.

Even though it is not mandatory to assign a weighting coefficient to the data present in the base, it is still preferable, in order to increase the quality of the comparison.

But it is however possible to envisage databases with only two inputs:
an input corresponding to the task or the stimulus applied
an input corresponding to the normalized data.

These examples below describe a particular embodiment of implementation of the invention, on the basis of an MRI analysis of a task performed with a block paradigm.

However, a person skilled in the art will be able to adapt the steps described hereinbelow in the case of an event-driven paradigm, of a subject in resting state, or with another data acquisition mode.

### DESCRIPTION OF THE FIGURES

FIG. 1: list of the 116 grey matter elements that can be used in a normalized atlas

FIG. 2: list of the 58 bundles of white matter that can be used in a normalized atlas

FIG. 3: algorithm for obtaining normalized data and for comparison with a database. FL: fuzzy logic; SB: white matter; SG: grey matter; Fx: bundles; Fa: anisotropic fraction; Nb: number; Lg: length; diff stat sign: significant statistical difference.

### EXAMPLES

### Example 1

### Data Acquisitions

1.1 Acquisitions of the functional brain data of the grey matter (cortex, central grey nuclei) during the performance of a cognitive task (block paradigm) in activation functional MRI among healthy subjects: spatial resolution: 2 mm³; temporal resolution: 1.5 s

1.2 Acquisition of the structural data of the white matter fibers linking the grey matter areas in diffusion tensor MRI (DT1 or HARDI technique); spatial resolution: 2 mm³

1.3 Acquisition of the anatomical data in T1-weighted volume morphological MRI; spatial resolution: 2 mm³

1.4 Paradigm used: 3 activation blocks in the paradigm with 360 inputs (acquisitions) per block

### Example 2

### Analysis of the Data Acquired

2.1 Co-registration and normalization of the anatomical data on the T1 atlas of the MNI (spatial resolution: 1 mm³). After correction of the movement artefacts and of the spatial deformations due to the MRI acquisition methods used (echo planar), co-registration and normalization of the functional and structural data on the anatomical data of the subject already co-registered on the atlas of the MNI; spatial resolution of all the data: 1 mm³

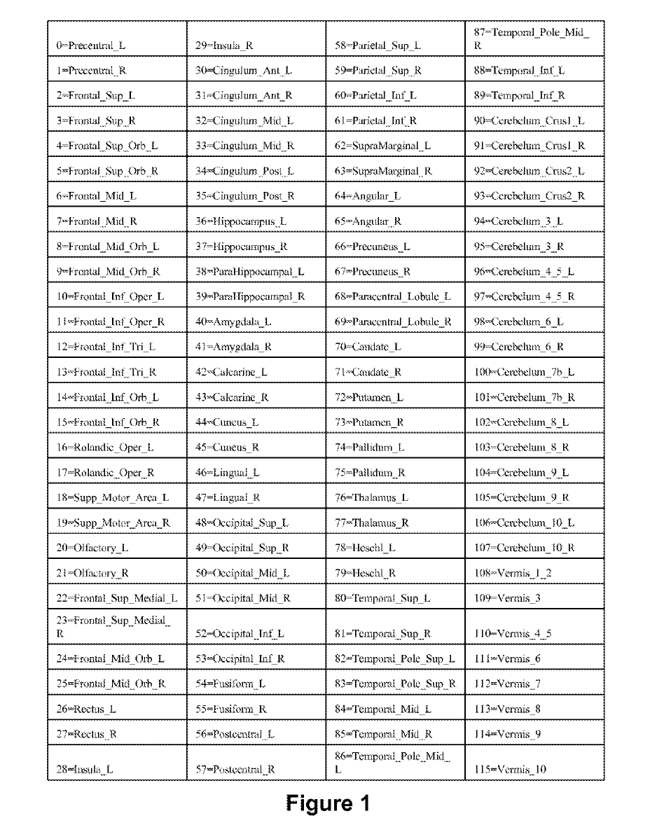

2.2 Functional data (grey matter): after new co-registration and spatial normalization of the data acquired on a specific anatomical atlas with 116 inputs (cortical areas and central grey nuclei, FIG. 1), analysis in pseudo-real time (temporal resolution of the acquisition interpolated with an algorithm, making it possible to switch from a resolution of 1.5 s to 150 ms) of the statistically significant signal variations at the level of each voxel acquired in activation fMRI during the task performed according to a block or event-driven paradigm, by using the statistical general linear model, and by normalizing the results in a coordinate with fixed temporal resolution (150 ms).

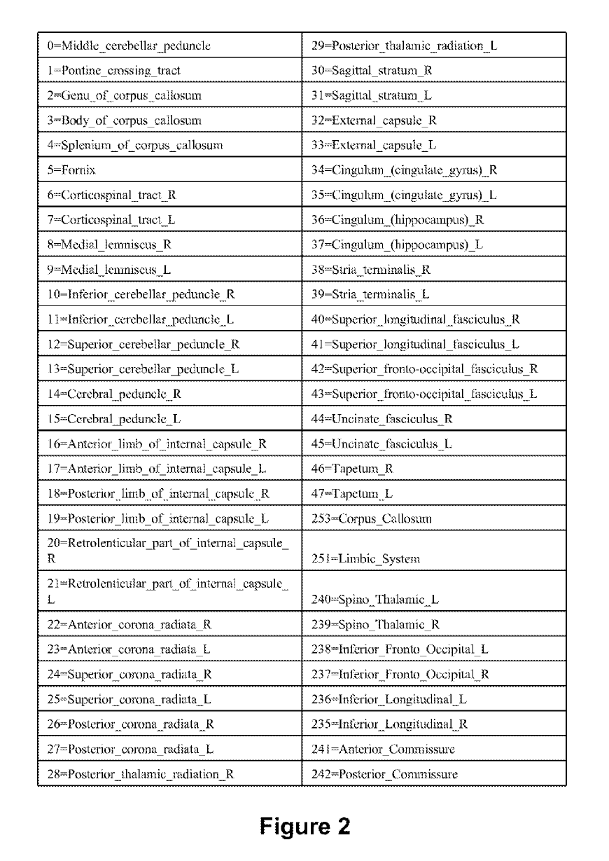

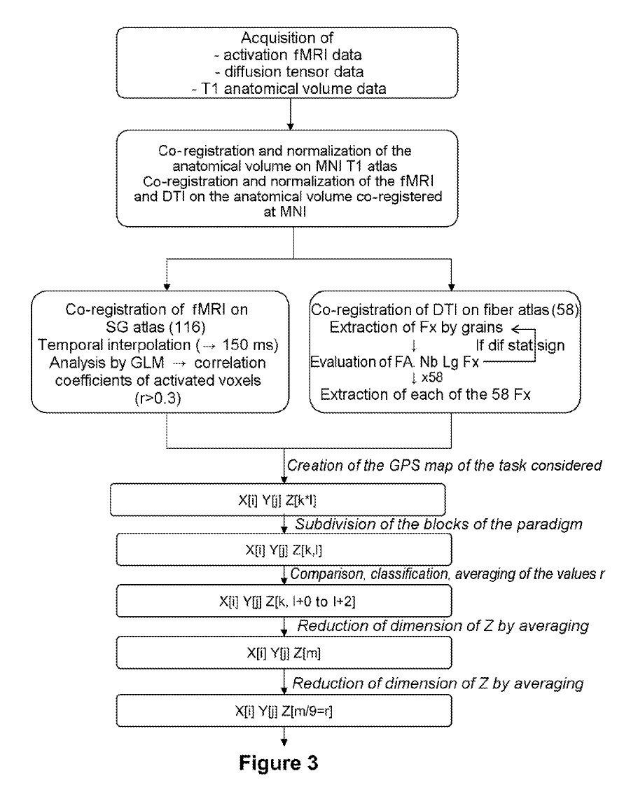

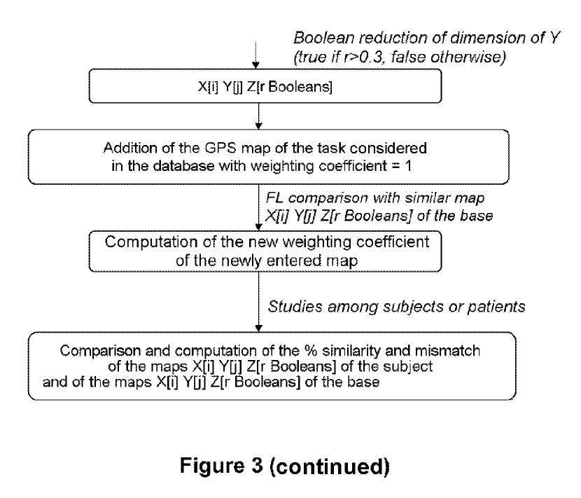

2.3 Structural data (white matter): the data acquired in diffusion tensor already co-registered on the anatomical atlas used in 2.1 and 2.2 are again co-registered and normalized on an atlas of the specific white matter fibers comprising 58 bundles (details in FIG. 2). The extraction of the bundles of the subject studied is performed automatically by using a mid-deterministic, mid-probabilistic global tractography method.

The extraction is performed as follows: the atlas initiates the extraction algorithm by supplying the extraction departure areas (grains) for each of the 58 bundles considered, and by iteratively comparing the results of the extraction obtained (parameters analyzed: anisotropy fraction, bundle length, number of fibers) with the known values of the initial bundle in the atlas. The iterations are stopped when the statistical differences between these values are no longer significant, and/or when there is overlapping of bundles.

### Example 3

### Analysis of the Structural and Functional Connections: Establishment of the “GPS Maps” for the Brain Task Studied

A first analysis is performed per temporal block of 150 ms over the set of the blocks of the paradigm of the functional acquisition.

For each temporal block, the algorithm searches voxel by voxel, for the activated functional brain areas, and searches for the bundles of fibers uniting each of these activated brain areas. The mapping is established according to a Boolean model (activated, not activated) per cortical region, per white matter bundle, and per temporal block. The parameter for individualizing the activated areas is set according to a statistical threshold of r=0.3.

A statistical classification with weighted geometrical averaging of the results obtained within each temporal block is then performed by comparing the results obtained in each block of the paradigm with those of the other blocks of the paradigm so as to normalize the

“GPS” mapping thus created.

A reduction of the temporal dimension of this map is performed by averaging of the results of the temporal blocks by a factor 9.

In particular

Map dimensions and inputs:

SG=grey matter

SB=white matter

r: coefficient of correlation with the paradigm

1^{st} Analysis

X[nb SG areas]×Y[nb SB bundles]×Z[(nb activated paradigms*nb temporal blocks within the paradigm)]

For a paradigm of 3 activated paradigm blocks, and 360 temporal blocks of 150 ms within the paradigm:

X[116]×Y[58]×Z[3*360]

Each input at X[i], Y[j], Z[k*l] is retained if their r>0.3 (significance threshold), and not retained (set to the value=0) otherwise.

2^{nd} Analysis

Subdivision of X[116]×Y[58]×Z[3*360] into X[116]×Y[58]×Z[3,360] (over Z 3 temporal areas of 360 inputs). This step in fact corresponds to recognizing that, in a paradigm of 3 activated paradigm blocks of a duration T, there are three identical blocks present and switching from a state reflecting this duration T to three reflecting states each of a duration T/3, each corresponding to one of the activated paradigm blocks.

Comparison, classification and averaging between
X[i], Y[j], Z[k,l]
X[i], Y[j], Z[k,l+1] and
X[i], Y[j], Z[k.l+2], with 0≦1≦2

Creation of a map X[i]. Y[j], Z[m] with m=k=average of the results between Z[k,l], Z[k.l+1] and Z[k,l+2]

3. Reduction of the Dimensions of the Map

Transformation of the map X[116]×Y[58]×Z[3*360] into map X[116]×Y[58]×Z[360] (averaging between each block of the paradigm)

then into X[116]×Y[58]×Z[40] (reduction by a temporal factor 9)

by averaging of i=0 to 8 of the X[116]×Y[58]×Z[i]

4. Boolean Normalization of the Map

The values are retained if r average >0.3, and are not retained otherwise.

### Example 4

### Recording of the Data in a Database

Each map established for a specific task is inserted into an input of the database, with an initial weighting coefficient set at 1.

The database comprises 3 inputs with different dimensions:
n maps of a given cognitive task
n weighting coefficients, and
m numbers of different cognitive tasks.

On each new input into the base, if there are already one or more similar task maps inserted into the base, the weighting coefficient of the new map inserted is recomputed by using a fuzzy logic algorithm making it possible to compute the percentage similarity between the new task to be inserted into the base and the data already present.

The analysis of the weighting coefficient is performed by comparing the X[116]×Y[58]×Z[40] values of each map with one another and by determining their similarity threshold.

Thus, the more different inputs the database has for a task merit, the more accurate the weighting coefficient of the new input will be.

This database becomes increasingly relevant as it is enriched with new inputs.

### Example 5

### Use of the Reference Database

For each functional task performed by a subject (or patient), the map generated is compared to the inputs of the base by using the same fuzzy logic algorithm as that used for the computation of the weighting coefficients during the creation and the enrichment of the database.

It is possible to compare a specific task with the similar tasks already present in the database (e.g.: motricity of the left hand); the result of this comparison will then be a percentage similarity on the specific task studied.

It is possible to compare a specific task with different tasks already present in the database (e.g.:

motricity of the left hand versus the right hand; motricity of the mouth versus verbal fluency by category, etc.). There will then be a percentage similarity available which will take into account the areas common to two different tasks and a percentage mismatch which will take into account the brain areas not common to the two different tasks.

1. A method for generating normalized data that can be used for the implementation of a method as claimed in one of claims 1 to 4, in which said data comprise, for each temporal acquisition block (t1) of the data during the performance of the task or the application of the stimulus, the protocol for performing said task or for applying said stimulus representing a paradigm:
a. of the functional brain data of the grey matter obtained during the performance of said cognitive task or after application of the stimulus
b. of the structural data of the white matter fibers linking the grey matter areas, obtained during the performance of said cognitive task or after application of the stimulus
c. of the anatomical data of the brain of said subject,
said method comprising the steps of
i. normalization of said anatomical data, in order to represent the brain of the subject in a normalized coordinate
ii. normalization of said functional data on the basis of a normalized functional atlas representing the cortical areas and the central grey nuclei of the brain, said functional atlas being in the same normalized coordinate as the atlas of (i)
iii. normalization of said structural data of the white matter fibers on the basis of said anatomical atlas of (ii)
iv. for each temporal block (t1), searching, voxel by voxel, for the activated functional brain areas, and for the bundles of white matter fibers uniting each of these activated brain areas
v. for each of the functional brain areas studied, averaging of the correlation coefficients of each of the voxels of said area with the paradigm, in order to obtain a unique correlation coefficient of said area with the paradigm
said normalized data obtained on completion of the step v thus consisting of:
for each temporal block (t1)
normalized data representing the white matter fibers of the brain of the subject
normalized data representing the correlation coefficient of each functional area (grey matter) with the paradigm (performance of the task or application of the stimulus)
definition of the task performed or of the stimulus applied.

2. The method as claimed in claim 2, further comprising a step of increasing the temporal resolution between each temporal acquisition block (t1), by dividing into equal parts (interpolated temporal blocks (t2)) the time between two temporal acquisition blocks (t1) and interpolating the statistically significant signal variations at the level of each voxel acquired for the functional data by using a generalized linear model, said step being performed between the step (iii) of normalization of the structural data and the step (iv) of searching, voxel by voxel, for the activated functional brain areas, and for the bundles of white matter fibers uniting each of these activated brain areas,
said step (iv) being then performed for each interpolated temporal block (t2),
said normalized data obtained on completion of the step v thus consisting of:
for each temporal block (t2)
d. normalized data representing the white matter fibers of the brain of the subject
e. normalized data representing the correlation coefficient of each functional area (grey matter) with the paradigm (performance of the task or application of the stimulus)
f. definition of the task performed or of the stimulus applied.

3. The method as claimed in claim 1 or 2, characterized in that said functional brain areas studied in the step v are only the areas for which a voxel-by-voxel search has identified that they are activated.

4. The method as claimed in one of claims 1 to 3, further comprising a step of averaging of the results of the temporal blocks corresponding to the paradigm, said step making it possible to obtain normalized data consisting of
normalized data representing the white matter fibers of the brain of the subject
normalized data representing the correlation coefficient of each functional area (grey matter) with the paradigm (performance of the task or application of the stimulus)
definition of the task performed or of the stimulus applied.

5. The method as claimed in one of claims 1 to 4, further comprising a storage of said data within a database.

6. The method as claimed in claim 5, characterized in that, upon the storage of said data in the base, a weighting coefficient is assigned to said data, computed by using a fuzzy logic algorithm, by comparing said data to be stored to those already contained in the base, for said task or said stimulus.

7. A method for analyzing the brain activity of a subject during the performance of a task or in response to a stimulus comprising the steps of
normalization of data (d1) collected during the performance of said task or application of said stimulus in order to obtain normalized data (d2), by implementation of the method as claimed in one of claims 1 to 6,
comparison of said normalized data (d2) with data (d3) present in a normalized database
said data (d3) of said database each being specific to a task of a given stimulus, said comparison being performed by a fuzzy logic algorithm, said method making it possible to determine a degree of similarity of said normalized data (d2) with data present in the normalized database,
said method making it possible to determine the brain activity of said subject during the performance of said task or in response to said stimulus.

8. The method as claimed in claim 7, characterized in that said data (d1) collected during the performance of the task or the application of the stimulus are MRI, PET scanner, echography, or electroencephalography (EEG) data.

9. The use of the method as claimed in one of claim 7 or 8, in a comparative analysis of the state of wakefulness at rest among healthy subjects and among subjects who cannot communicate with the environment.

10. The use of the method as claimed in one of claim 7 or 8, in an analysis of psychiatric problems in a subject.

11. The use of the method as claimed in one of claim 7 or 8, in a clinical and functional evaluation of a deficiency or of a neurological problem in a subject.

12. The use of the method as claimed in one of claim 7 or 8, for detecting whether a subject tells the truth or lies in responding to different questions.

13. The use of the method as claimed in one of claim 7 or 8, in neuromarketing studies, for better understanding the reactions of subjects upon the presentation of new products, or the evocation of product development.

14. The use of the method as claimed in one of claim 7 or 8, characterized in that said stimulus is an absence of stimulus and that the patient is at rest (resting state).
